(19) United States
(12) Patent Application Publication
(10) Pub. No.: US 2023/0125213 A1
(43) Pub. Date: Apr. 27, 2023

(54) HANDHELD ADAPTER FOR ROBOTIC SURGICAL INSTRUMENTS

(71) Applicant: Distalmotion SA, Epalinges (CH)

(72) Inventors: Julien CHASSOT, Lechelles (CH); Michael FRIEDRICH, Bern (CH)

(73) Assignee: Distalmotion SA, Epalinges (CH)

(21) Appl. No.: 18/049,915

(22) Filed: Oct. 26, 2022

Related U.S. Application Data
(60) Provisional application No. 63/263,145, filed on Oct. 27, 2021.

(51) Int. Cl. A61B 1/313; A61B 34/35; A61B 34/00

(52) U.S. Cl. CPC A61B 1/3132; A61B 34/35; A61B 34/75; A61B 2017/00477

(57) ABSTRACT

The present disclosure provides handheld adapters allowing the use of robotic surgical instruments during standard laparoscopic phases. The handheld adapter includes a plurality of receptacles for engaging with a plurality of engagers of the surgical instrument to thereby actuate an end-effector of the surgical instrument in the close degrees of freedom via a plurality of force transmitting elements upon actuation of a trigger. The handheld adapter further includes a spring coupled to the plurality of receptacles and configured to provide a predetermined tension on the plurality of force transmitting elements, such that upon release of the trigger, the spring causes movement of the plurality of receptacles, which causes movement of the respective engagers to thereby actuate the end-effector in the open degree of freedom.

### CROSS-REFERENCE TO RELATED APPLICATIONS

This application claims priority to U.S. Provisional Patent Application Ser. No. 63/263,145, filed Oct. 27, 2021, the entire contents of which are incorporated herein by reference.

### FIELD OF USE

The present disclosure is directed to handheld adapters for releasably coupling to surgical instruments for performing laparoscopic procedures.

### BACKGROUND

Numerous environments and applications call for remote actuation with teleoperated surgical devices. These applications include the ability to perform fine manipulation, to manipulate in confined spaces, manipulate in dangerous or contaminated environments, in clean-room or sterile environments and in surgical environments, whether open field or minimally invasive. While these applications vary, along with parameters such as precise tolerances and the level of skill of the end user, each demands many of the same features from a teleoperated system, such as the ability to carry out dexterous manipulation with high precision.

Surgical applications are discussed in the following disclosure in more detail as exemplary of applications for a teleoperated device system where known devices exist but significant shortcomings are evident in previously-known systems and methods.

Open surgery is still the preferred method for many surgical procedures. It has been used by the medical community for many decades and typically required making long incisions in the abdomen or other area of the body, through which traditional surgical tools are inserted. Due to such incisions, this extremely invasive approach results in substantial blood loss during surgery and, typically, long and painful recuperation periods in a hospital setting.

Laparoscopy, a minimally invasive technique, was developed to overcome some of the disadvantages of open surgery. Instead of large through-wall incisions, several small openings are made in the patient through which long and thin surgical instruments and endoscopic cameras are inserted. The minimally invasive nature of laparoscopic procedures reduces blood loss and pain and shortens hospital stays. When performed by experienced surgeons, a laparoscopic technique can attain clinical outcomes similar to open surgery. However, despite the above-mentioned advantages, laparoscopy requires a high degree of skill to successfully manipulate the rigid and long instrumentation used in such procedures. Typically, the entry incision acts as a point of rotation, decreasing the freedom for positioning and orientating the instruments inside the patient. The movements of the surgeon's hand about this incision point are inverted and scaled-up relative to the instrument tip (“fulcrum effect”), which reduces dexterity and sensitivity and magnifies any tremors of the surgeon's hands. In addition, the long and straight instruments force the surgeon to work in an uncomfortable posture for hands, arms and body, which can be tremendously tiring during a prolonged procedure. Therefore, due to these drawbacks of laparoscopic instrumentation, minimally invasive techniques are mainly limited to use in simple surgeries, while only a small minority of surgeons is able to use such instrumentation and methods in complex procedures.

To overcome the foregoing limitations of previously-known systems, surgical robotic systems were developed to provide an easier-to-use approach to complex minimally invasive surgeries. By means of a computerized robotic interface, those systems enable the performance of remote laparoscopy where the surgeon sits at a console manipulating two master manipulators to perform the operation through several small incisions. Like manual laparoscopy, the robotic approach is also minimally invasive, providing the above-mentioned advantages over open surgery with respect to reduced pain, blood loss, and recuperation time. In addition, it also offers better ergonomy for the surgeon compared to open and laparoscopic techniques, improved dexterity, precision, and tremor suppression, and the removal of the fulcrum effect. Accordingly, the surgical robot system provides the value of robotics for long and difficult surgical tasks such as suturing and dissection.

However, during certain surgical operations, a surgeon may need to intervene at a moment's notice to perform integrated laparoscopy for short and specialized surgical tasks such as vessel sealing and stapling. To address this, Dexter®, a hybrid surgical robot system made available by Distalmotion SA of Epalinges, Switzerland, permits a surgeon to efficiently switch between robotic laparoscopy and integrated laparoscopy for short and specialized surgical tasks as described in U.S. Pat. No. 10,413,374 to Chassot, the entire contents of which is incorporated by reference herein. Thus, hybrid surgical robot systems may require the use of both standard and robotic laparoscopic tools during the same operation to provide the best outcome for the surgical procedure, which therefore requires duplicated costly instruments used during one single operation.

Accordingly, it would be desirable to provide handheld surgical systems that may be used with the same interchangeable surgical instruments utilized by the robotic telemanipulators of a surgical robot system to permit the surgeon to seamlessly switch between the surgical robot system and manual laparoscopy, and thereby reduce cost by requiring less varying instrumentation.

### SUMMARY

The present disclosure overcomes the drawbacks of previously-known systems and methods by providing a handheld adapter for actuating a surgical instrument having an end-effector. The handheld adapter may include a frame having an instrument connection interface sized and shaped to receive the surgical instrument therethrough, and a plurality of receptacles for engaging with respective engagers of a plurality of engagers of the surgical instrument to actuate the end effector in a close degree of freedom via a first pair of force transmitting elements of the surgical instrument and to actuate the end effector in an open degree of freedom via a second pair of force transmitting elements of the surgical instrument. The handheld adapter further may include a handle having a trigger that may be actuated to cause movement of the plurality of receptacles, which causes movement of the respective engagers to thereby actuate the end-effector in the close degree of freedom. In addition, the handheld adapter may include a spring coupled to the frame and the plurality of receptacles to provide a predetermined tension on the first and second pair of force transmitting elements when the surgical instrument is received by the frame, such that upon release of the trigger, the spring causes movement of the plurality of receptacles, which causes movement of the respective engagers to thereby actuate the end-effector in the open degree of freedom.

The instrument connection interface may include a locking mechanism for locking the surgical instrument relative to the frame. For example, the locking mechanism may include a circumferential groove sized and shaped to rotatably receive a locking pin of the surgical instrument. In addition, the handheld adapter may include an opening sized and shaped to receive an instrument shaft of the surgical instrument therethrough, the opening having a geometry that facilitates the transmission of torque to the instrument shaft of the surgical instrument.

In some embodiments, the plurality of receptacles includes a first pair of receptacles and a second pair of receptacles, such that the first pair of receptacles engages with a first pair of engagers of the plurality of engagers of the surgical instrument to actuate the end-effector in the close degree of freedom via the first pair of force transmitting elements of the surgical instrument, and the second pair of receptacles engages with a second pair of engagers of the plurality of engagers of the surgical instrument to actuate the end-effector in the open degree of freedom via the second pair of force transmitting elements of the surgical instrument. Accordingly, the spring may be coupled to the second pair of receptacles, such that upon release of the trigger, the spring causes translational movement of the second pair of receptacles, which causes translational movement of the second pair of engagers to thereby actuate the end-effector in the open degree of freedom.

Moreover, translational movement of the first pair of engagers causes translational movement of the second pair of engagers in an equal and opposite direction via the first and second pair of force transmitting elements, such that, upon actuation of the trigger, the translational movement of the second pair of engagers causes translational movement of the second pair of receptacles, which causes the spring to compress axially. In addition, the second pair of receptacles may include a hook shape to interengage with the second pair of engagers of the surgical instrument. The handheld adapter further may include a slider slidably moveable within the frame, wherein the second pair of receptacles extends from the slider, and wherein the spring is coupled to the second pair of receptacles via the slider. The handheld adapter also may include a third pair of receptacles that engages with a third pair of engagers of the surgical instrument to prevent actuation of the third pair of engagers.

In accordance with another aspect of the present disclosure, a surgical instrument for coupling to the handheld adapters described herein is provided. The surgical instrument may include an instrument shaft having an end effector, and a surgical instrument interface having a first pair of engagers for engaging with the first pair of receptacles of the handheld adapter to actuate the end effector in the close degree of freedom, and a second pair of engagers for engaging with the second pair of receptacles to actuate the end effector in the open degree of freedom. The surgical instrument further may include a first pair of force transmitting elements extending through the instrument shaft and coupled to the first pair of engagers and the end effector, and a second pair of force transmitting element extending through the instrument shaft and coupled to the second pair of engagers and the end effector, wherein movement of the first pair of force transmitting elements causes a corresponding movement of the second pair of force transmitting elements in an equal and opposite direction.

In accordance with another aspect of the present disclosure, the plurality of receptacles of the handheld adapter may include a pair of receptacles that engage with a pair of engagers of the plurality of engagers of the surgical instrument, such that movement of the pair of receptacles in a first direction actuates the end-effector in a close degree of freedom via the first pair of force transmitting elements of the surgical instrument, and movement of the pair of receptacles in a second direction opposite to the first direction actuates the end-effector in an open degree of freedom via the second pair of force transmitting elements of the surgical instrument. Accordingly, the spring may be coupled to the pair of receptacles, such that upon release of the trigger, the spring causes movement of the pair of receptacles in the second direction, which causes movement of the second pair of engagers in the second direction to thereby actuate the end-effector in the open degree of freedom.

In some embodiments, the handheld adapter may include a rack slidably moveable within the frame, and the plurality of receptacles may include a pair of pinions that rotatably engage with the rack. Accordingly, the spring may be coupled to the plurality of receptacles via the rack. In addition, the pair of engagers may include a pair of spools for engaging with the pair of pinions when the surgical instrument is received by the frame. Thus, actuation of the trigger causes translational movement of the rack, which causes rotational movement of the pair of engagers in the first direction to thereby actuate the end-effector in the close degree of freedom. Translational movement of the rack in the first direction causes the spring to compress axially, such that, upon release of the trigger, the spring causes movement of the pair of receptacles in the second direction via the rack.

In some embodiments, the handheld adapter may include a slider slidably moveable within the frame, the slider having the pair of receptacles. Accordingly, the spring may be coupled to the pair of receptacles via the slider. The pair of receptacles may include a pair of grooves sized and shaped to receive the pair of engagers therein when the surgical instrument is received by the frame. Thus, actuation of the trigger causes translational movement of the slider, which causes translational movement of the pair of engagers in the first direction to thereby actuate the end-effector in the close degree of freedom. Translational movement of the slider in the first direction causes the spring to compress axially, such that, upon release of the trigger, the spring causes movement of the pair of receptacles in the second direction via the slider.

### BRIEF DESCRIPTION OF THE DRAWINGS

FIG. 1 illustrates an exemplary system for laparoscopic procedures constructed in accordance with the principles of the present disclosure.

FIGS. 2A-2D illustrates an exemplary surgical instrument constructed in accordance with the principles of the present disclosure.

FIG. 3 illustrates an exemplary handheld adapter constructed in accordance with the principles of the present disclosure.

FIGS. 4A and 4B illustrate coupling of a surgical instrument to the handheld adapter of FIG. 3.

FIG. 4C illustrates force transmission between a surgical instrument and the handheld adapter of FIG. 3 in accordance with the principles of the present disclosure.

FIG. 5A illustrates force transmission between an alternative surgical instrument and an alternative exemplary handheld adapter in accordance with the principles of the present disclosure.

FIG. 5B illustrates the instrument interface of the surgical instrument of FIG. 5A.

FIG. 6A illustrates force transmission between another alternative surgical instrument and another alternative exemplary handheld adapter in accordance with the principles of the present disclosure.

FIG. 6B illustrates the instrument interface of the surgical instrument of FIG. 6A.

### DETAILED DESCRIPTION

The present disclosure provides handheld adapters allowing the use of robotic surgical instruments during standard laparoscopic phases. The handheld adapters reduce the need to have additional standard laparoscopic instruments during the operation, thereby reducing the overall cost of the consumables per surgery. The handheld adapters also simplify the flow when electrosurgical instruments are used both in standard laparoscopic and robotic phases, eliminating the need to connect/disconnect the instruments and to adjust the electrosurgical generator settings.

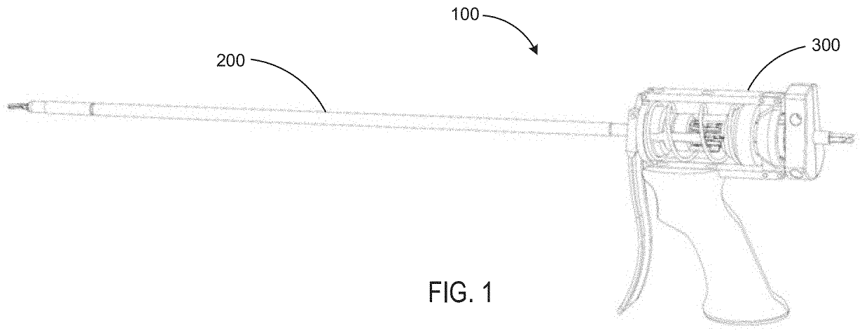

Referring now to FIG. 1, an exemplary system for performing laparoscopic procedures is provided. System 100 includes handheld adapter 300, which may be removably coupled to surgical instrument 200. System 100 may be operated by a user, e.g., a surgeon, during laparoscopic phases of an operation with a hybrid robotic approach. Accordingly, surgical instrument 200 may be coupled to handheld adapter 300 when needed to perform manual standard laparoscopy, and decoupled and removed from handheld adapter 300 when needed to perform robotic surgery. For example, surgical instrument 200 may be coupled to surgical robotic systems such as those described in U.S. Pat. No. 10,413,374 to Chassot, the entire contents of which are incorporated herein by reference, for performing robotic surgical procedures.

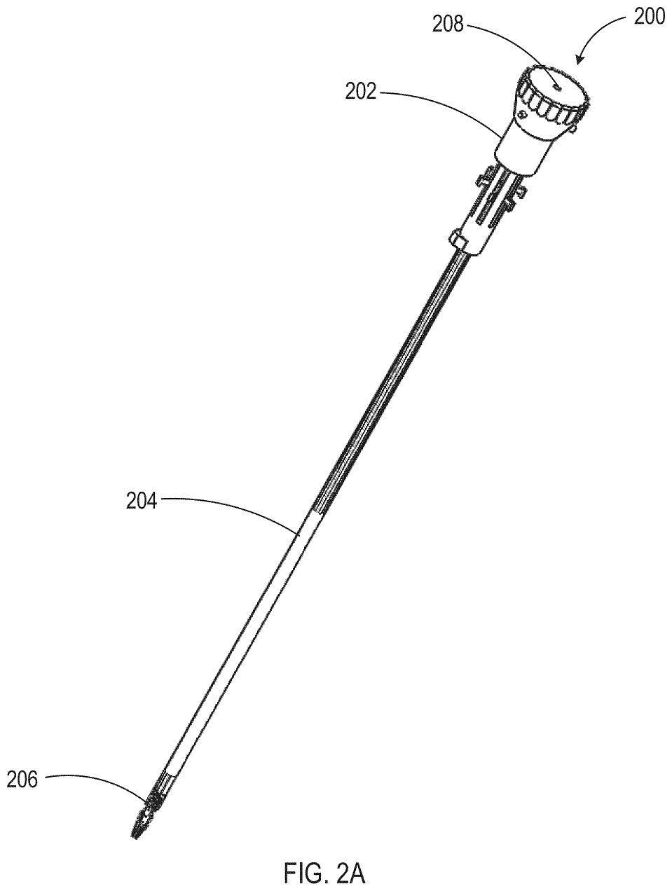

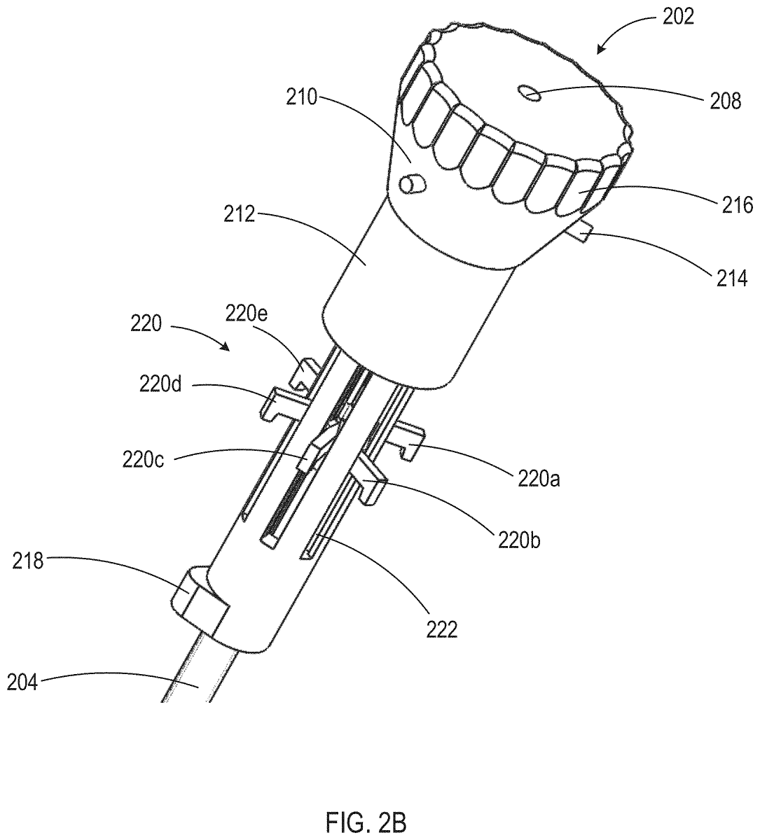

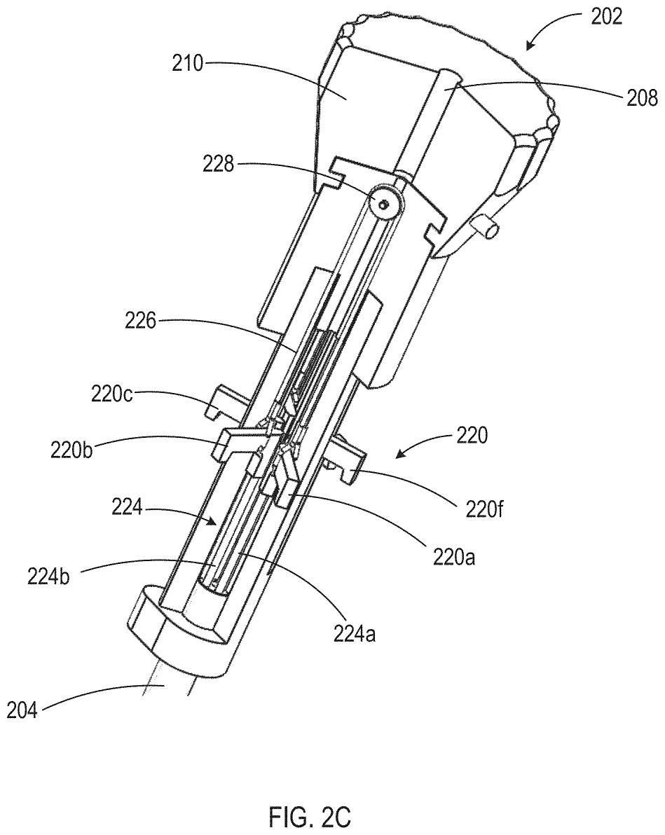

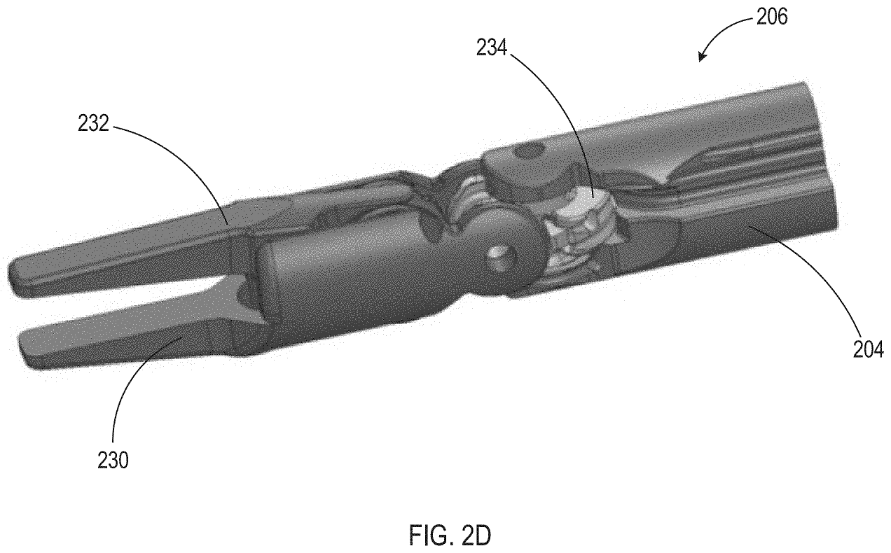

Referring now to FIGS. 2A to 2D, an exemplary surgical instrument constructed in accordance with one aspect of the present disclosure is provided. Surgical instrument 200 may be constructed as described in U.S. Pat. No. 11,058,503 to Chassot, the entire contents of which are incorporated herein by reference. As shown in FIG. 2A, instrument 200 illustratively includes head 202 at a proximal region of instrument 200, end-effector 206 at a distal region of instrument 200 and shaft 204, which is preferably elongated, extending therebetween. Instrument 200 also may include lumen 208 extending through head 202 and shaft 204. Instrument 200 is sized and shaped to be inserted through an opening of handheld adapter 300 and engaged with handheld adapter 300 such that a force by handheld adapter 300 may be translationally transmitted to instrument 200 to actuate movement of end-effector 206 in one or more degrees-of-freedom, as described in further detail below. Instrument 200 may be reusable but is preferably disposable after a single use.

Referring now to FIGS. 2B and 2C, head 202 is described in further detail. As shown in FIG. 2B, head 202 includes lumen 208 extending therethrough. Lumen 208 may be sized and shaped to receive electrical cables electrically coupled to the electrosurgical generators when end-effector 206 has electrosurgical instruments. Head 202 may have rotatable portion 210 and fixed portion 212. Rotatable portion 210 rotates relative to fixed portion 212 about the longitudinal axis of instrument 200. Rotatable portion 210 may include locking pins 214 sized and shaped to enter a corresponding groove of the instrument connection interface of adapter 300, such that rotation of rotatable portion 210 causes locking pins 214 to enter the groove and secure instrument 200 within adapter 300. As will be understood by one skilled in the art, locking pins 214 may have any shape that may effectively secure instrument 200 within handheld adapter 300. Rotatable portion 210 may have grooves 216 along the surface of rotatable portion 210 such that an operator of teleoperated surgical instrument 10 may achieve an enhanced grip and rotate rotatable portion 210 easier. Head 202 further may include key 218, e.g., a puka-yoke, shaped and sized such to ensure proper axial alignment of instrument 200 within handheld adapter 300.

In some embodiments, head 202 may include an identification tag, e.g., RFID or barcode, configured to store information regarding instrument 200, e.g., instrument type, serial number, calibration data, range-of-motion, end-effector kinematics such as numbers and types of degrees-of-freedom including serial-serial, serial-parallel, yaw-pitch-actuate, pitch-yaw-actuate, roll-pitch-yaw-actuate, pitch-roll-actuate, etc., or controlling offsets. Such instrument information may be detected from the identification tag via a control system of the teleoperated surgical instrument by scanning the identification tag and/or electrically coupling the teleoperated surgical instrument to instrument 200.

Head 202 preferably includes plurality of engagers 220 permitted to move translationally responsive to user input at adapter 300 to actuate movement at the end-effector in one or more degrees-of-freedom. For example, plurality of engagers 220 may independently move translationally along corresponding linear pathways 222 (e.g., slot in the proximal region of the shaft) responsive to translational movement at corresponding receptacles of adapter 300 coupled thereto caused by user input at adapter 300. Engagers 220 are sized and shaped to contact the corresponding receptacles of adapter 300. For example, engagers 220 may have a hook shape, or any other shape understood in the art to maximize transmission of force between engagers 220 and the corresponding receptacles of adapter 300. Engagers 220 may be coupled to end-effector 206 via a plurality of force transmitting elements disposed within lumen 208 of shaft 204, as described in further detail below. When actuated, engagers 220 apply force to end-effector 206 via the force transmitting element(s) to move end-effector 206 in at least one degree of freedom. For example, actuator 220 may move in a translational manner, e.g., in a direction parallel to the longitudinal axis of elongated shaft 204, which in turn moves end-effector 206 via the force transmitting element couple therebetween.

As shown in FIG. 2C, each of engagers 220 may be coupled to force transmitting elements 224, e.g., rigid elements such as steel, composite or polymeric rods, flexible elements such as tungsten, steel, polymer, or Dyneema cables, wires or ropes, or semi-rigid elements such as a metal band, at one end of force transmitting elements 224. The other end of force transmitting elements 224 may be coupled to a component of end-effector 206 such that actuation of engagers 220 actuates movement of end-effector 206 in one of three degrees-of-freedom.

Plurality of engagers 220 may include multiple pairs of engagers, e.g., engagers 220a and 220b, engagers 220c and 200d, and engagers 200e and 200f, each pair of engagers coupled to respective components of end-effector 206 via respective pairs of force transmitting elements to move end-effector 206 in a respective degree of freedom. Accordingly, translational movement of a first engager of the pair of engagers causes a corresponding, equal and opposite translational movement of the second engager of the pair of engagers via the respective pair of force transmitting elements. For example, pair of engagers 220a and 200b may be coupled to end-effector 206 via pair of force transmitting elements 224a and 224b, respectively, to actuate end-effector 206 in a first degree of freedom responsive to force applied at engagers 220a and 200b. Similarly, pairs of engagers 200c and 200d may be coupled to end-effector 206 via a second pair of force transmitting elements to actuate end-effector 206 in a second degree of freedom, and pairs of engagers 200e and 200f may be coupled to end-effector 206 via a third pair of force transmitting elements to actuate end-effector 206 in a third degree of freedom. In some embodiments, plurality of engagers 220 may include a fourth pair of engagers coupled to end-effector 206 via a fourth pair of force transmitting elements to actuate end-effector 206 in a fourth degree of freedom, e.g., rotation.

Prior to insertion of instrument 200 into handheld adapter 300, instrument 200 may maintain a minimum “off-use” tension to keep force transmitting elements 224 in their proper pathway and prevent unraveling. For example, a minimum “off-use” tension may be achieved by closing the loop of force transmitting elements 224, e.g., pair of force transmitting elements 224a, 224b, by applying a force to plurality of engagers 220, e.g., pair of engagers 220a and 220b, via cable 226, e.g., a metallic or polymeric cable, and pulley 228 disposed within head 202. Pulley 228 may be disposed toward rotatable portion 210 of head 202 such that cable 226 extends from one of the engagers, e.g., engager 220a, over pulley 228, to the other engager of pair of engagers 220, e.g., engager 220b.

Referring now to FIG. 2D, an exemplary end-effector is provided. End-effector 206 may be coupled to the distal end of instrument shaft 204, such that plurality of force transmitting elements 224 may extend through the lumen of instrument shaft 204 and coupled to respective components of end-effector 206. As shown in FIG. 2D, end-effector 206 may include first jaw 230, second jaw 232, and yaw component 234. Pair of engagers 220a and 220b may be coupled to first jaw 230 via pair of force transmitting elements 224a and 224b for actuating first jaw 230 in a first degree of freedom, pairs of engagers 220c and 220d may be coupled to second jaw 232 via second pair of force transmitting elements 224c and 224d for actuating second jaw 232 in a second degree of freedom, and pairs of engagers 220e and 220f may be coupled to yaw component 234 via pair of force transmitting elements 224e and 224f for actuating yaw component 234 in a third degree of freedom, e.g., yaw.

Accordingly, actuation of pair of engagers 200a and 200b may be selectively coordinated with actuation of pair of engagers 200c and 200d to actuate end-effector 206 in an articulation degree of freedom, e.g., open and close, and a pitch degree of freedom. For example, actuation of first jaw 230 via pair of engagers 200a and 200b and second jaw 232 via pair of engagers 200c and 200d in the same direction will cause end-effector 206 to move in the pitch degree of freedom. Moreover, actuation of first jaw 230 via pair of engagers 200a and 200b and second jaw 232 via pair of engagers 200c and 200d in opposite directions will cause end-effector 206 to either open or close, e.g., move away from each other or move toward each other. Actuation of yaw component 234 via pair of engagers 220e and 200f will cause end-effector 206 to move in the yaw degree of freedom.

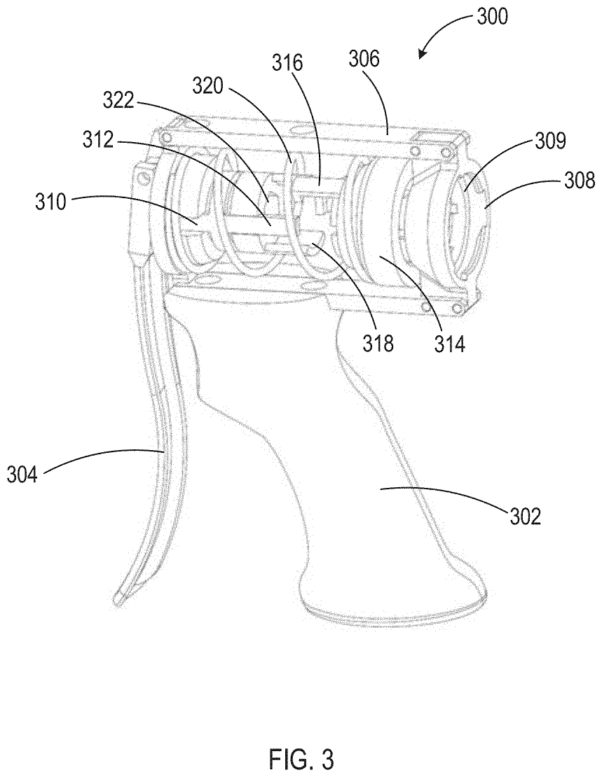

Referring now to FIG. 3, an exemplary handheld adapter constructed in accordance with one aspect of the present disclosure is provided. Handheld adapter 300 may be removably coupled to a surgical instrument, e.g., surgical instrument 200, for allowing a surgeon to perform laparoscopic procedures. Accordingly, adapter 300 may include handle 302 sized and shaped for a surgeon to grasp with their hand, frame 306 having an opening extending therethrough for receiving surgical instrument 200, and trigger 304 operatively coupled to frame 306.

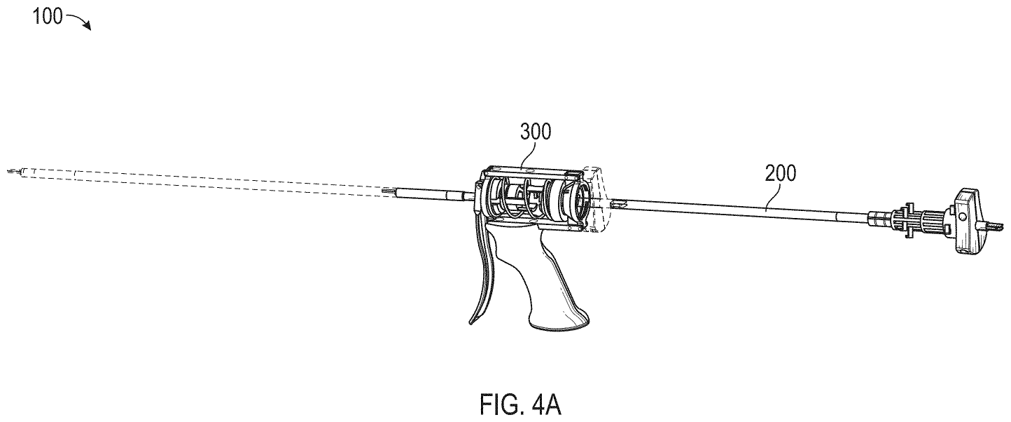

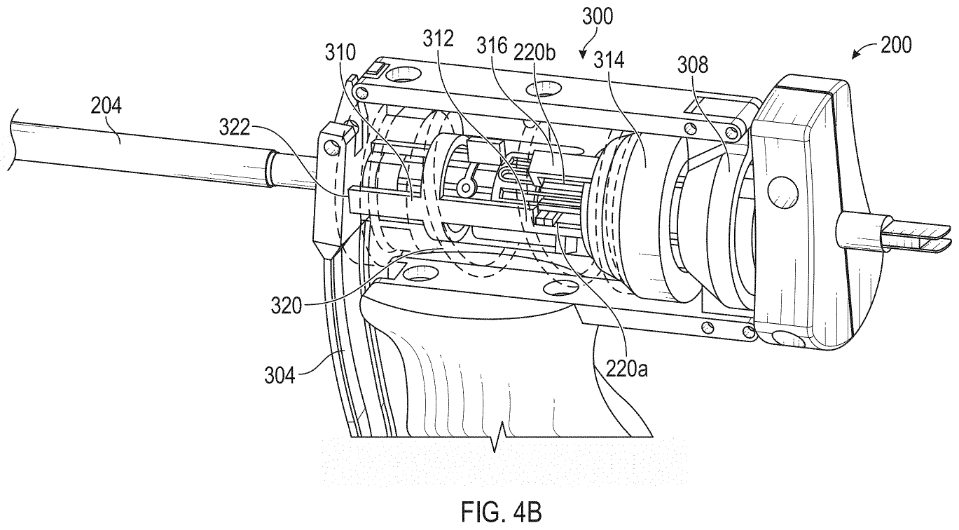

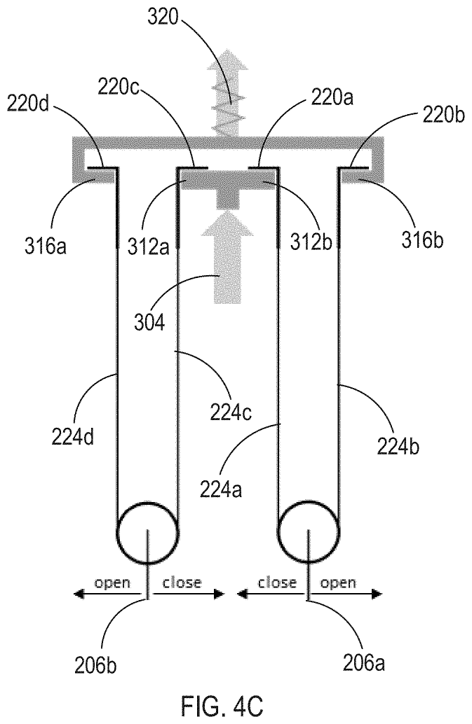

As shown in FIG. 3, frame 306 may include instrument connection interface 308 having an opening sized and shaped to receive at least a portion of surgical instrument 200 therethrough. Insertion of surgical instrument 200 within handheld adapter 300 is illustrated in FIG. 4A, engagement of surgical instrument 200 with handheld adapter 300 is illustrated in FIG. 4B, and force transmission between handheld adapter 300 and surgical instrument 200 is illustrated in FIG. 4C. Interface 308 may include a locking mechanism to secure surgical instrument 200 in place relative to frame 306 when surgical instrument is inserted through interface 308, as shown in FIG. 4A.

Referring again to FIG. 3, as described above, surgical instrument 200 may include locking pins 214, and thus, interface 308 may include one or more grooves 309 sized and shaped to rotatably receive locking pins 214 therein to thereby secure surgical instrument 200 to frame 306. Moreover, frame 306 may include opening 322 sized and shaped for receiving instrument shaft 204 therethrough. Opening 322 may have a geometry selected to facilitate the transmission of torque to instrument shaft 204, e.g., during actuation of a rotation degree of freedom of end-effector 206. For example, opening 322 may have a hexagonal shape, or any non-circular shape.

In addition, frame 306 may include pair of receptacles 312 extending from pair of sliders 310 for engaging with plurality of engagers 220, e.g., pair of engagers 220a, 220c, to actuate the close degree of freedom of end-effector 206. For example, as shown in FIG. 4C, pair of receptacles 312, e.g., receptacles 312a and 312b, may be engaged with engagers 220c and 200a, respectively, when surgical instrument 200 is coupled to handheld adapter 300. As shown in FIG. 3, pair of sliders 310 may be coupled to each other via a ring having an opening sized and shaped to receive at least a portion of surgical instrument 200 therethrough. As shown in FIG. 4B, trigger 304 may be operatively coupled to slider 310 via connection point 322. Accordingly, upon actuation of trigger 304, slider 310, and accordingly receptacles 312a, 312b, moves translationally relative to frame 306, which causes translational movement of engagers 220c, 220a when surgical instrument 200 is coupled to handheld adapter 300.

As shown in FIG. 3, frame 306 further may include pair of receptacles 316 extending from slider 314 for engaging with plurality of engagers 220, e.g., pair of engagers 220b, 220d, to actuate the open degree of freedom of end-effector 206. For example, as shown in FIG. 4C, pair of receptacles 316, e.g., receptacles 316a and 316b, may be engaged with engagers 220d and 200b, respectively, when surgical instrument 200 is coupled to handheld adapter 300. As shown in FIG. 3, slider 314 may be slidably disposed within frame 306, and may include an opening to permit at least a portion of surgical instrument 200 to extend therethrough.

Preferably, receptacles 316 may have a hook-shape for interengaging with engagers 220b, 220d, such that translational movement of engagers 220b, 220d in one direction causes a corresponding translational movement of receptacles 316 in the same direction, e.g., via a pushing force, and translational movement of receptacles 316 in an opposite direction causes a corresponding translational movement of receptacles 316 in the same opposite direction, e.g., via a pulling force. As described above, pair of engagers 220a and 220b move translationally in equal and opposite directions via pair of force transmitting elements 224a and 224b, and pair of engagers 220c and 220d move translationally in equal and opposite directions via pair of force transmitting elements 224c and 224d. Accordingly, upon actuation of trigger 304, which causes translational movement of engagers 220a, 220c in a first direction when surgical instrument 200 is coupled to handheld adapter 300, engagers 220b, 220d move translationally in a second direction equal and opposite to the first direction of engagers 220a, 220c, which causes translational movement of receptacles 316b, 316a, e.g., via a pushing force. The simultaneous actuation of engagers 220a, 220c provided by handheld adapter 300 permits end-effector 206 to effectively open and close in a precise, controlled manner.

As shown in FIG. 3, handheld adapter 300 further may include spring 320 disposed within frame 306. Spring 320 may be fixed to frame 306 at one end, and coupled to slider 314 at its other end. Spring 320 may be pre-loaded with a predetermined tension, such that spring 320 provides tension on the force transmitting elements when surgical instrument 200 is coupled to handheld adapter 300. Accordingly, upon actuation of trigger 304, which causes translational movement of receptacles 316a, 316b, and accordingly slider 314, in an equal and opposite direction to the translational movement of receptacles 312a, 312b, spring 320 compresses axially. This actuates end-effector 206 in the close degree of freedom. Specifically, as shown in FIG. 4C, translational movement of engagers 220a, 220c, causes first jaw 206a and second jaw 206b of end-effector 206, respectively, to move toward each other via force transmitting elements 224a, 224b, respectively, thereby actuating end-effector 206 in the close degree of freedom.

Upon release of trigger 304, spring 320 returns to its natural state, thereby applying a force to slider 314 which causes translational movement of slider 314, and accordingly receptacles 316a, 316b, which further causes translational movement of engagers 220d, 220b, e.g., via a pulling force, thereby actuating end-effector 206 in the open degree of freedom. Specifically, as shown in FIG. 4C, translational movement of engagers 220b, 220d, causes first jaw 206a and second jaw 206b of end-effector 206, respectively, to move away from each other via force transmitting elements 224b, 224d, respectively, thereby actuating end-effector 206 in the open degree of freedom. The pre-loaded tension of spring 320 may be selected such that end-effector 206 is biased in the open configuration upon engagement of surgical instrument 200 and handheld adapter 300.

Referring again to FIG. 3, frame 306 further may include receptacle 318 sized and shaped to engage with and prevent movement of pair of engagers 220e, 200f, which are responsible for actuating end-effector 206 in the yaw degree of freedom, as described above. As shown in FIG. 3, receptacle 318 may be a single component that engages both engagers 200e, 200f simultaneously, or alternatively, receptacle 318 may be formed of two components, each individually engaging engagers 200e, 200f, respectively. Accordingly, handheld adapter 300 prevents actuation of end-effector 206 in the yaw degree of freedom. Moreover, as handheld adapter 300 actuates engagers 220a, 220c simultaneously, handheld adapter 300 prevents actuation of end-effector 206 in the pitch degree of freedom.

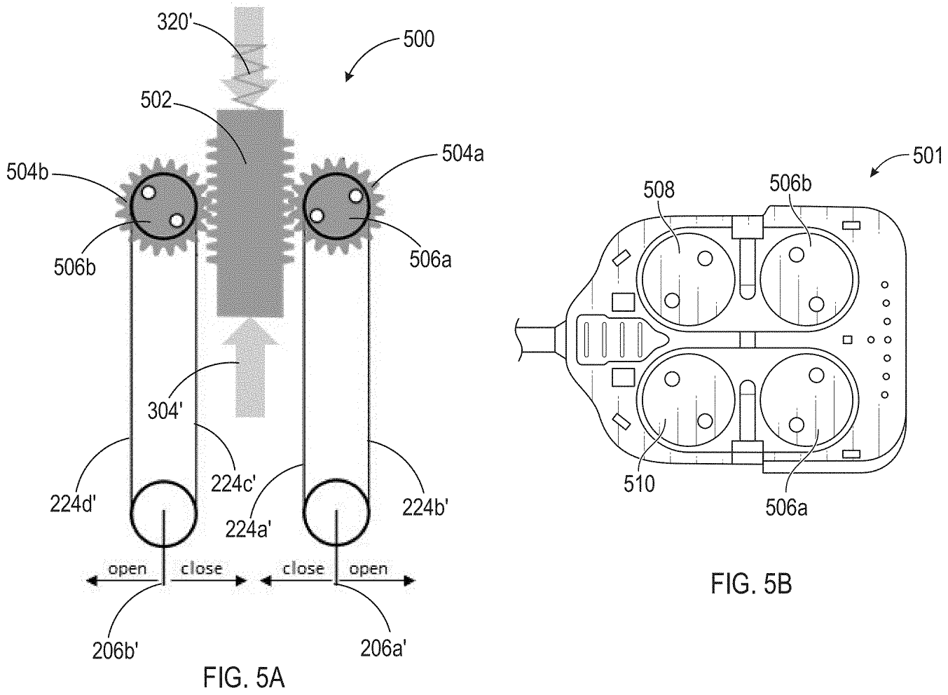

As will be understood by a person having ordinary skill in the art, the handheld adapter described herein may be adapted to engage with and actuate various surgical instruments in accordance with the principles of the present disclosure. For example, as shown in FIGS. 5A and 5B, exemplary handheld adapter 500 may be adapted to engage with and actuate surgical instrument 501, which may be constructed as described in U.S. Pat. No. 6,331,181, assigned to Intuitive Surgical, Inc., the entire contents of which is incorporated herein by reference. As shown in FIG. 5A, many components of surgical instrument 501 may be constructed similar to surgical instrument 200 of FIGS. 2A to 2D, with similar components having like-prime reference numerals. For example, force transmitting elements 224a′, 224b′, 224c′, 224d′ corresponds with force transmitting elements 224a, 224b, 224c, 224d, respectively, first jaw 206a′ and second jaw 206b′ corresponds with first jaw 206a and second jaw 206b, respectively, trigger 304′ corresponds with trigger 304, and spring 320′ correspond with spring 320.

Surgical instrument 501 differs from surgical instrument 200 in that, instead of a plurality of engagers coupled to the proximal ends of force transmitting elements 224a′, 224b′, 224c′, 224d′, the proximal ends of force transmitting elements 224a′, 224b′, 224c′, 224d′ are coupled to a pair of engagers, e.g., pair of spools 506a, 506b. Specifically, force transmitting elements 224a′, 224b may be coupled to spool 506a, and force transmitting elements 224c′, 224d′ may be coupled to spool 506b. As further shown in FIG. 5A, instead of receptacles 220a, 220b, 220c, 220d, handheld adapter 500 includes plurality of receptacles, e.g., pair of pinions 504a, 504b, sized and shaped to engage with spools 506a, 506b, respectively. Thus, upon engagement of surgical instrument 501 with handheld adapter 500, spool 506a engages with pinion 504a, and spool 506b engages with pinion 504b. As shown in FIGS. 5A, spring 320′ may be operatively coupled to pair of pinions 504a, 504b, via rack 502.

As shown in FIG. 5A, at least a portion of the outer circumference of pinions 504a, 504b may have a toothed geometry, e.g., gear-shaped. Moreover, handheld adapter 500 may include rack 502, having an outer surface geometry that engages with the outer circumference of pinions 504a, 504b. Accordingly, rack 502 and pinions 504a, 504b engage as a rack and pinion mechanism. Rack 502 is operatively coupled to trigger 304′. Thus, upon actuation of trigger 304′, rack 502 moves translationally in a first direction, which causes pinions 504a, 504b to rotate, e.g., in opposite directions. For example, as shown in FIG. 5A, translational movement of rack 502 in the first direction causes pinion 504a, and accordingly spool 506a, to rotate clockwise, and causes pinion 504b, and accordingly spool 506b, to rotate counter-clockwise, via force transmitting elements 224a′, 224b′ and force transmitting elements 224c′, 224d′, respectively, the simultaneous actuation of which causes first jaw 206a′ and second jaw 206b′ to move toward each other, to thereby actuate the end-effector in the close degree of freedom.

Like handheld adapter 300, upon actuation of trigger 304′, spring 320′ compresses axially due to the translational movement of rack 502 in the first direction. Thus, upon release of trigger 304′, spring 320′ returns to its natural state, thereby applying a force to rack 502 which causes translational movement of rack 502 in a second direction opposite to the first direction, which further causes pinion 504a, and accordingly spool 506a, to rotate in the counter-clockwise direction and pinion 504b, and accordingly spool 506b, to rotate in the clockwise direction, via the rack and pinion mechanism, thereby actuating the end-effector in the open degree of freedom. Specifically, as shown in FIG. 5A, counter-clockwise rotation of spool 506a and clockwise rotation of spool 506b, causes first jaw 206a′ and second jaw 206b′ of the end-effector, respectively, to move away from each other via force transmitting elements 224b′, 224a′ and force transmitting elements 224d′, 224c′, respectively, thereby actuating the end-effector in the open degree of freedom. The pre-loaded tension of spring 320′ may be selected such that the end-effector is biased in the open configuration upon engagement of surgical instrument 501 and handheld adapter 500.

Moreover, like handheld adapter 300, handheld adapter 500 may include one or more receptacles for engaging with and preventing movement the spool of surgical instrument 501 which is responsible for actuating the end-effector in the pitch degree of freedom, e.g., spool 508 as shown in FIG. 5B, and one or more receptacles for engaging with and preventing movement of the spool of surgical instrument 501 which is responsible for actuating the end-effector in the roll degree of freedom. Accordingly, handheld adapter 500 prevents actuation of the end-effector in the pitch and roll degrees of freedom. Moreover, as handheld adapter 500 actuates spools 504a, 504b simultaneously, handheld adapter 500 prevents actuation of the end-effector in the yaw degree of freedom.

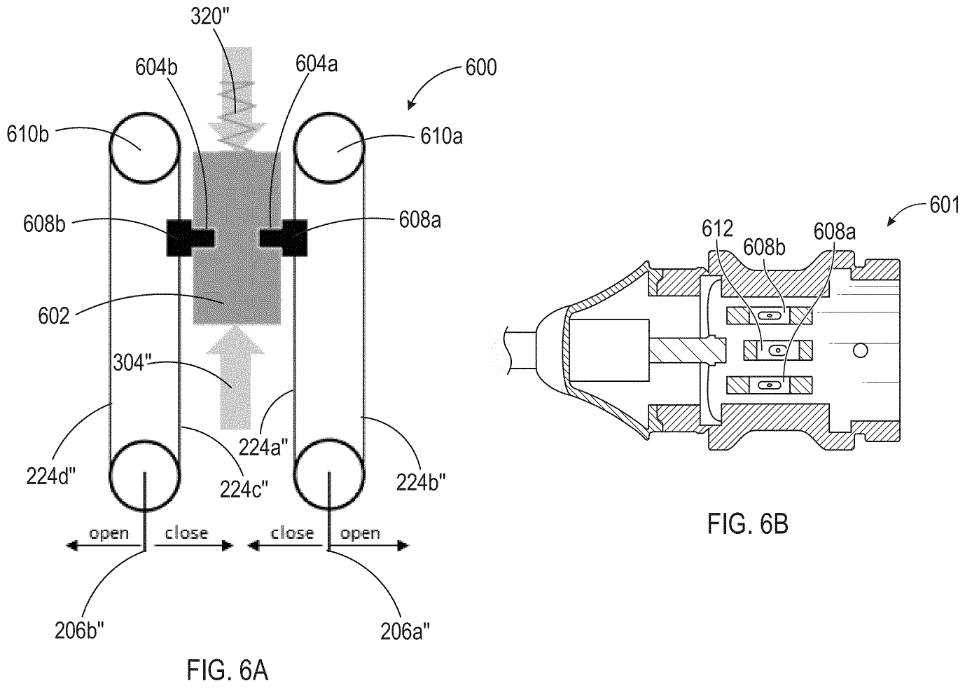

Referring now to FIGS. 6A and 6B, exemplary handheld adapter 600 may be adapted to engage with and actuate surgical instrument 601, which may be constructed as described in U.S. Pat. No. 10,792,113, assigned to CMR Surgical Limited, the entire contents of which is incorporated herein by reference. As shown in FIG. 6A, many components of surgical instrument 601 may be constructed similar to surgical instrument 200 of FIGS. 2A to 2D, with similar components having like-double prime reference numerals. For example, force transmitting elements 224a″, 224b″, 224c″, 224d″ corresponds with force transmitting elements 224a, 224b, 224c, 224d, respectively, first jaw 206a″ and second jaw 206b″ corresponds with first jaw 206a and second jaw 206b, respectively, trigger 304″ corresponds with trigger 304, and spring 320″ correspond with spring 320.

Surgical instrument 601 differs from surgical instrument 200 in that, instead of a plurality of engagers coupled to the proximal ends of force transmitting elements 224a″, 224b″, 224c″, 224d″, the proximal ends of force transmitting elements 224a′, 224b′, 224c′, 224d′ are coupled to a pair of pulleys 610a, 610b, thereby forming a closed loop with first jaw 206a″ and second jaw 206b″, respectively. As shown in FIG. 6A, force transmitting cables 224a″ and 224c″ are coupled to a pair of engagers 608a, 608b, respectively. As further shown in FIG. 6A, instead of receptacles 220a, 220b, 220c, 220d, handheld adapter 600 includes slider 602, which has a pair of receptacles, e.g., grooves 604a, 604b, disposed on its outer surface, and sized and shaped to engage with engagers 608a, 608b, respectively. Thus, upon engagement of surgical instrument 601 with handheld adapter 600, groove 604a engages with engager 608a, and groove 604b engages with engager 608b. As shown in FIGS. 6A, spring 320″ may be operatively coupled to pair of grooves 604a, 604b, via slider 602.

Slider 602 is operatively coupled to trigger 304″. Thus, upon actuation of trigger 304″, slider 602 moves translationally in a first direction, which causes translational movement of engagers 608a, 608b in the first direction via grooves 604a, 604b, the simultaneous actuation of which causes first jaw 206a″ and second jaw 206b″ to move toward each other, to thereby actuate the end-effector in the close degree of freedom. Specifically, translational movement of engagers 608a, 608b in the first direction causes a corresponding movement of force transmitting elements 224a″, 224c″, respectively, in the first direction, which causes a corresponding equal movement of force transmitting elements 224b″, 224d″ in a second direction opposite to the first direction via pulleys 610a, 610b, respectively, which causes first jaw 206a″ and second jaw 206b″ to move towards each other.

Like handheld adapter 300, upon actuation of trigger 304″, spring 320″ compresses axially due to the translational movement of slider 602 in the first direction. Thus, upon release of trigger 304″, spring 320″ returns to its natural state, thereby applying a force to slider 602 which causes translational movement of slider 602 in the second direction opposite to the first direction, which further causes translational movement of pair of engagers 608a, 608b in the second direction via grooves 604a, 604b, thereby actuating the end-effector in the open degree of freedom. Specifically, as shown in FIG. 6A, movement of engagers 608a, 608b in the second direction causes a corresponding movement of force transmitting elements 224a″, 224c″, respectively, in the second direction, which causes a corresponding equal movement of force transmitting elements 224b″, 224d″ in the first direction opposite to the second direction via pulleys 610a, 610b, respectively, which causes first jaw 206a″ and second jaw 206b″ to move away from each other, thereby actuating the end-effector in the open degree of freedom. The pre-loaded tension of spring 320″ may be selected such that the end-effector is biased in the open configuration upon engagement of surgical instrument 601 and handheld adapter 600.

Moreover, like handheld adapter 300, handheld adapter 600 may include one or more receptacles for engaging with and preventing movement the engager of surgical instrument 601 which is responsible for actuating the end-effector in the pitch degree of freedom, e.g., engager 612 as shown in FIG. 6B. Accordingly, handheld adapter 600 prevents actuation of the end-effector in the pitch degree of freedom. Moreover, as handheld adapter 600 actuates engagers 604a, 604b simultaneously, handheld adapter 600 prevents actuation of the end-effector in the yaw degree of freedom.

While various illustrative embodiments of the invention are described above, it will be apparent to one skilled in the art that various changes and modifications may be made therein without departing from the invention. The appended claims are intended to cover all such changes and modifications that fall within the true scope of the invention.

What is claimed:

1. A handheld adapter for actuating a surgical instrument having an end-effector, the handheld adapter comprising:
a frame comprising an instrument connection interface sized and shaped to receive the surgical instrument;
a plurality of receptacles configured to engage with respective engagers of a plurality of engagers of the surgical instrument to actuate the end effector in a close degree of freedom via a first pair of force transmitting elements of the surgical instrument and to actuate the end effector in an open degree of freedom via a second pair of force transmitting elements of the surgical instrument;
a handle comprising a trigger configured to be actuated to cause movement of the plurality of receptacles, which causes movement of the respective engagers to thereby actuate the end-effector in the close degree of freedom; and
a spring coupled to the frame and the plurality of receptacles and configured to provide a predetermined tension on the first and second pair of force transmitting elements when the surgical instrument is received by the frame, such that upon release of the trigger, the spring causes movement of the plurality of receptacles, which causes movement of the respective engagers to thereby actuate the end-effector in the open degree of freedom.

2. The handheld adapter of claim 1, wherein the instrument connection interface comprises a locking mechanism configured to lock the surgical instrument relative to the frame.

3. The handheld adapter of claim 2, wherein the locking mechanism comprises a circumferential groove sized and shaped to rotatably receive a locking pin of the surgical instrument.

4. The handheld adapter of claim 1, further comprising an opening sized and shaped to receive an instrument shaft of the surgical instrument therethrough, the opening comprising a geometry configured to transmit torque to the instrument shaft of the surgical instrument.

5. The handheld adapter of claim 1, wherein the plurality of receptacles comprises a first pair of receptacles and a second pair of receptacles, the first pair of receptacles configured to engage with a first pair of engagers of the plurality of engagers of the surgical instrument to actuate the end-effector in the close degree of freedom via the first pair of force transmitting elements of the surgical instrument, and the second pair of receptacles configured to engage with a second pair of engagers of the plurality of engagers of the surgical instrument to actuate the end-effector in the open degree of freedom via the second pair of force transmitting elements of the surgical instrument.

6. The handheld adapter of claim 5, wherein the spring is coupled to the second pair of receptacles, such that upon release of the trigger, the spring causes translational movement of the second pair of receptacles, which causes translational movement of the second pair of engagers to thereby actuate the end-effector in the open degree of freedom.

7. The handheld adapter of claim 5, wherein translational movement of the first pair of engagers causes translational movement of the second pair of engagers in an equal and opposite direction via the first and second pair of force transmitting elements, such that, upon actuation of the trigger, the translational movement of the second pair of engagers causes translational movement of the second pair of receptacles, which causes the spring to compress axially.

8. The handheld adapter of claim 5, wherein the second pair of receptacles comprises a hook shape configured to interengage with the second pair of engagers of the surgical instrument.

9. The handheld adapter of claim 5, further comprising a slider slidably moveable within the frame, wherein the second pair of receptacles extends from the slider, and wherein the spring is coupled to the second pair of receptacles via the slider.

10. The handheld adapter of claim 5, further comprising a third pair of receptacles configured to engage with a third pair of engagers of the surgical instrument to prevent actuation of the third pair of engagers.

11. A surgical instrument configured to be coupled to and actuated via the handheld adapter of claim 6, the surgical instrument comprising:
an instrument shaft having an end effector;
a surgical instrument interface comprising a first pair of engagers configured to engage with the first pair of receptacles of the handheld adapter to actuate the end effector in the close degree of freedom, and a second pair of engagers configured to engage with the second pair of receptacles to actuate the end effector in the open degree of freedom;
a first pair of force transmitting elements extending through the instrument shaft and coupled to the first pair of engagers and the end effector; and
a second pair of force transmitting element extending through the instrument shaft and coupled to the second pair of engagers and the end effector,
wherein movement of the first pair of force transmitting elements causes a corresponding movement of the second pair of force transmitting elements in an equal and opposite direction.

12. The handheld adapter of claim 1, wherein the plurality of receptacles comprises a pair of receptacles configured to engage with a pair of engagers of the plurality of engagers of the surgical instrument, such that movement of the pair of receptacles in a first direction actuates the end-effector in a close degree of freedom via the first pair of force transmitting elements of the surgical instrument, and movement of the pair of receptacles in a second direction opposite to the first direction actuates the end-effector in an open degree of freedom via the second pair of force transmitting elements of the surgical instrument, and
wherein the spring is coupled to the pair of receptacles, such that upon release of the trigger, the spring causes movement of the pair of receptacles in the second direction, which causes movement of the second pair of engagers in the second direction to thereby actuate the end-effector in the open degree of freedom.

13. The handheld adapter of claim 12, further comprising a rack slidably moveable within the frame, wherein the plurality of receptacles comprises a pair of pinions configured to rotatably engage with the rack, and wherein the spring is coupled to the plurality of receptacles via the rack.

14. The handheld adapter of claim 13, wherein the pair of engagers comprises a pair of spools configured to engage with the pair of pinions when the surgical instrument is received by the frame.

15. The handheld adapter of claim 13, wherein actuation of the trigger causes translational movement of the rack, which causes rotational movement of the pair of engagers in the first direction to thereby actuate the end-effector in the close degree of freedom.

16. The handheld adapter of claim 15, wherein translational movement of the rack in the first direction causes the spring to compress axially, such that, upon release of the trigger, the spring causes movement of the pair of receptacles in the second direction via the rack.

17. The handheld adapter of claim 12, further comprising a slider slidably moveable within the frame, the slider comprising the pair of receptacles, and wherein the spring is coupled to the pair of receptacles via the slider.

18. The handheld adapter of claim 17, wherein the pair of receptacles comprises a pair of grooves sized and shaped to receive the pair of engagers therein when the surgical instrument is received by the frame.

19. The handheld adapter of claim 17, wherein actuation of the trigger causes translational movement of the slider, which causes translational movement of the pair of engagers in the first direction to thereby actuate the end-effector in the close degree of freedom.

20. The handheld adapter of claim 19, wherein translational movement of the slider in the first direction causes the spring to compress axially, such that, upon release of the trigger, the spring causes movement of the pair of receptacles in the second direction via the slider.
